(19) United States
(12) Patent Application Publication
(10) Pub. No.: US 2002/0128346 A1
(43) Pub. Date: Sep. 12, 2002

(54) Hydrogels

(76) Inventors: Angelika Maria Domschke, Duluth, GA (US); Vimala Mary Francis, Suwanee, GA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/755,357

(22) Filed: Jan. 5, 2001

Related U.S. Application Data
(60) Provisional application No. 60/174,458, filed on Jan. 5, 2000.

(51) Int. Cl. A61F 2/00; C08K 3/00

(52) U.S. Cl. 523/113; 524/803; 523/114

(57) ABSTRACT

The present invention relates to a cell growth substrate polymer that is obtainable by polymerizing a polymerizable component comprising least one hydrophilic monomer or macromer which is devoid of a sulfo group, at least one sulfo-group-containing monomer, and optionally a crosslinker in a weight ratio as defined in the claims. The polymers of the invention are useful, for example, as substrates for the attachment and growth of mammalian cells and tissue and in particular as materials for the manufacture of biomedical devices and prostheses, including implanted devices.

The present invention relates to a hydrogel system that incorporates specific sulfonates for cell growth stimulation, its preparation and use for various biomedical applications.

There is extensive teaching in the literature about the interaction of tissue with the surfaces of synthetic polymer materials that are intended for use in implants. Much of this teaching arises from research that has aimed at the design of polymer surfaces that would support the very tight and effective attachment of tissue cells to the polymeric surface. Such polymer surfaces are intended for certain demanding implant applications, such as the tissue-contacting surfaces of percutaneous access devices. Another such application would be for use as the lumenal surface of small diameter vascular grafts, where it is intended that endothelial cells would cover the polymer surface. In these applications, tight binding of the cells to the surface of the synthetic polymer is required for the implant to be effective.

It has generally been thought that the adhesion of cells to synthetic polymeric substrates requires the surface chemistry or topography of the synthetic polymer to be specifically modified to facilitate the adhesion and growth of cells. Glow discharge, plasma polymerization and radiation grafting are a few of the techniques known in the art for such polymer modification it has also been described that cell attachment to surfaces of synthetic hydrophobic polymers can alternatively be stimulated by the absorption or covalent attachment onto the polymer surface of one or more cell adhesive molecules or fragments thereof, such as fibronectin, vitronectin, collagen, or the like.

Until now the practice in the implantation of artificial corneas for replacement of corneal tissue (i.e. stromal tissue) has involved the surgical technique of making an incision above the cornea then cutting a deep pocket behind the epithelial layer to remove the damaged cornea; the replacement cornea was slid into this pocket and the incision closed by suturing. In this case cell growth on the implant was not required, nor necessarily desirable. A recently proposed procedure for the correction of refractive errors is the implantation of a lens within the corneal epithelium. The implantation of such an intraepithelial lens would typically be conducted by removing the corneal epithelial cell layers of the cornea by scraping, then placing the synthetic lens directly onto and in intimate contact with the corneal tissue. The synthetic lens will be held in place during the period immediately after its placement either by the material characteristics of the synthetic lens allowing it to adhere to the underlying tissue, or by use of a biocompatible glue, or by suturing.

From the prior art it would be expected that to satisfy this requirement polymers will require a chemical surface modification to generate a wettable surface.

WO96/31548 discloses a class of hydrophobic materials based on perfluoroalkylpolyether primary monomers, which particularly in their porous and coated form can act as cell growth substrates and are suitable for use as biomaterials, particularly in ocular applications. The document also discloses perfluoroalkylpolyether-containing compositions copolymerized with comonomers. Currently available biocompatible polymers for use as cell growth substrates suffer limitations mainly due to their pronounced hydrophobicity; some of the disadvantages are fouling with proteinaceous, carbohydrate and other such materials, and expense associated with additional processing steps such as surface modification to enable the synthetic polymer to support the adhesion and growth of cells, since human cells generally show little tendency to grow evenly on the surface of articles made from polymeric materials. Porous materials, on the other hand, tend to irreversibly absorb proteins which affects the optical transparency of the materials; also the permeability of the polymers to proteins, nutrients and the like is often not completely satisfactory. In particular, the permeability to high molecular weight proteins (about 600000 Daltons and higher) is difficult to achieve with the prior art materials. Moreover, the optical quality of the known materials may be affected during handling under ambient air or in contact with the biological environment. Many of the above-mentioned problems could be overcome by the use of biocompatible hydrophilic polymers such as, for example, poly(HEMA) which are, however, in general known to have no noticeable cell growth capability at all.

Accordingly, the problem to be solved within the present invention is to provide hydrophilic polymers such as poly(HEMA) with the ability to stimulate cell growth in order to create novel valuable biomaterials. It has now surprisingly been found that this can be achieved by copolymerizing the underlying hydrophilic monomer, for example HEMA, with a monomer containing a sulfo group. This sulfo-modification also introduces successfully antifouling properties.

The present invention therefore in one aspect relates to a cell growth substrate polymer that is obtainable by polymerizing a polymerizable component comprising from about 30 to about 99% by weight of at least one hydrophilic monomer or macromer which is devoid of a sulfo group, from about 1 to about 70% by weight of at least one sulfo-group-containing monomer, and from 0 to about 20% by weight of at least one low molecular weight crosslinker.

As used herein, a suitable hydrophilic monomer or macromer which is devoid of a sulfo group denotes an ethylenically unsaturated monomer that typically yields as homopolymer a polymer that can absorb at least 10% by weight of water.

Examples of hydrophilic monomers are hydroxy-substituted C₁-C₄-alkyl acrylates and methacrylates, for example hydroxyethyl methacrylate (HEMA), hydroxyethyl acrylate or hydroxypropyl acrylate, acrylamide, methacrylamide, N-mono- and N,N-di-C₁-C₄-alkyl acrylamides and methacrylamides which may be hydroxy-substituted in the alkyl moiety, hydroxy-substituted C₁-C₄-alkylvinylethers, allyl alcohol, vinyl acetate, vinylically unsaturated carboxylic acids having a total of 3 to 5 carbon atoms, for example acrylic or methacrylic acid, N-vinylpyrrolidone and N-acryloylmorpholine. Preferred hydrophilic monomers are, for example, selected from the group consisting of hydroxyethylmethacrylate (HEMA), N-vinylpyrrolidone (NVP), acrylamide, N,N-dimethylacrylamide (DMA) and N-acryloylmorpholine. A preferred sulfo-free hydrophilic monomer is HEMA or a copolymer comprising HEMA and one or more of the above-mentioned monomers, for example NVP or DMA. Most preferably the hydrophilic monomer is HEMA.

A suitable hydrophilic macromer is, for example, a vinylfunctionalized polyvinyl alcohol, polyalkylene oxide or N-vinylpyrrolidone homo- or copolymer. The macromer may contain one or more than one ethylenically unsaturated double bonds. Preferred hydrophilic macromers are a vinylfunctionalized polyvinyl alcohol or polyethylene oxide, in particular a vinylfunctionalized polyvinyl alcohol, for example as described in U.S. Pat. No. 5,508,317, issued to Beat Müller on Apr. 16, 1996, which is incorporated herein by reference. The weight average molecular weight of the hydrophilic macromer may vary within wide limits; a suitable range is from about 2000 up to 1,000,000. Preferably, the hydrophilic macromer has a molecular weight of up to 300,000, especially up to approximately 100,000 and especially preferably from about 5000 to about 50,000.

A suitable sulfocontaining monomer is, for example, an ethylenically unsaturated compound having from 2 to 18 C-atoms which is substituted by a sulfo group or a suitable salt thereof. Examples are methallylsulfonic acid, styrenesulfonic acid, sulfopropylmethacrylate, sulfopropyl-acrylate, 2-acrylamido-2-methylpropanesulfonic acid, vinyl sulfonic acid, or a suitable salt thereof, for example an alkaline salt or ammonium salt, in particular the sodium or potassium salt. Preferred sulfomonomers are sodium methallylsulfonate, sodium styrenesulfonate, potassium sulfopropylmethacrylate or potassium sulfopropylacrylate.

A suitable low molecular weight crosslinker, if present, is, for example, a di- or polyvinylic crosslinking agent such as ethylenglycol diacrylate or dimethacrylate, di-, tri- or tetraethylenglycol diacrylate or dimethacrylate, allyl (meth)acrylate, a C₂-C₈-alkylene diacrylate or dimethacrylate, divinyl ether, divinyl sulfone, di- and trivinylbenzene, trimethylolpropane triacrylate or trimethacrylate, pentaerythritol tetraacrylate or tetramethacrylate, bisphenol A diacrylate or dimethacrylate, methylene bisacrylamide or -bismethacrylamide, ethylene bisacrylamide or ethylene bismethacrylamide, triallyl phthalate or diallyl phthalate. The average weight average molecular weight of the crosslinker is, for example, up to 1000, preferably up to 750 and most preferably up to 500. Preferred crosslinkers according to the invention are ethyleneglycol-dimethacrylate, pentaerythritoltetraacrylate or pentaerythritoltetramethacrylate. The cell growth substrate polymers of the invention preferably comprise a crosslinker; the crosslinker is present, for example, in an amount of from 0.1 to 20% by weight, preferably from 0.5 to 15% by weight, and in particular 1 to 10% by weight, in each case based on the total polymerizable component.

The cell growth substrate polymers of the invention are preferably prepared from a polymerizable component comprising 70-98% by weight of one or more hydrophilic monomers or macromers which are devoid of a sulfo group, 2-30% by weight of a sulfomonomer and 0-10 % by weight of a crosslinker.

The cell growth substrate polymers of the invention are even more preferably prepared from a polymerizable component comprising 70-95% by weight of one or more hydrophilic monomers or macromers which are devoid of a sulfo group, 5-20% by weight of a sulfomonomer and 1-10 % by weight of a crosslinker.

Preferably the cell growth substrate polymer is prepared from a polymerizable component comprising about 70 to about 98% by weight of one or more hydrophilic monomers selected from the group consisting of HEMA, N-vinylpyrrolidone, acrylamide, N,N-dimethylacrylamide and N-acryloylmorpholine, about 2 to about 30% by weight of a sulfonate-group-containing monomer selected from the group consisting of sodium methallylsulfonate, sodium styrenesulfonate, potassium sulfopropylmethacrylate and potassium sulfopropylacrylate, and about 0 to about 10% by weight of a crosslinker selected from the group consisting of ethyleneglycol-dimethacrylate, pentaerythritoltetraacrylate and pentaerythritoltetramethacrylate.

More preferably the cell growth substrate polymer is prepared from a polymerizable component comprising about 70 to about 95% by weight of one or more hydrophilic monomers selected from the group consisting of HEMA and N-vinylpyrrolidone, about 5 to about 20% by weight of a sulfonate-group-containing monomer selected from the group consisting of potassium sulfopropylmethacrylate and potassium sulfopropylacrylate, and about 0 to about 10% by weight of a crosslinker, selected from the group consisting of pentaerythritoltetraacrylate and pentaerythritoltetramethacrylate.

Most preferably the cell growth substrate polymer is prepared from a polymerizable component comprising about 70 to about 95% by weight of HEMA, about 5 to about 20% by weight of potassium sulfopropylmethacrylate and about 1 to about 10% by weight of pentaerythritoltetraacrylate.

It has also been found that unlike the attachment of endothelial cells and fibroblasts to synthetic polymers as described in the prior art, in the case of the polymers as herein defined the initial attachment of corneal epithelial cells is not dependent on the absorption of the glycoproteins fibronectin or vitronectin from the culture medium. The present findings show that the polymers as herein defined directly support adhesion of corneal epithelial cells. This therefore obviates any additional surface modification of the material.

In another aspect, this invention provides a material for the attachment and growth of human or animal cells in vitro, wherein the material comprises a cell growth substrate polymer as herein defined.

In another aspect, this invention provides a material for the attachment and growth of human or animal cells in vivo, wherein the material comprises a cell growth substrate polymer as herein defined.

The cell growth substrate polymers of the invention may be obtained from the above-mentioned polymerizable component in conventional manner, for example by copolymerizing the hydrophilic monomer(s) or macromer(s) that are devoid of a sulfo group, the sulfomonomer(s) and optionally the crosslinker(s) and optionally solvent(s) and/or further additives to afford a transparent polymer.

Useful solvents include those selected from the following classes: water, esters, alkanols, ethers, halogenated solvents and mixtures thereof, preferably water, a C₁-C₄-alkylester of a C₂-C₄-carboxylic acid such as for example ethyl acetate, a C₁-C₄-alkanol such as for example methanol, ethanol or n-or isopropanol, and mixtures thereof, more preferably water, a C₁-C₂-alkanol and mixtures thereof, most preferably water, methanol and mixtures thereof. For the polymerization process water is the most desirable solvent.

Suitable further additives are, for example, a polymerization initiator, in case of the preferred photochemical initiation of the polymerizable component a photoinitiator, or is a suitable porogen providing porosity of the polymer, for example an optionally substituted poly(alkylene)glycol or a poly N-vinylpyrrolidone.

Examples of suitable photoinitiators are familiar to the person skilled in the art. Useful photoinitiators include for example benzophenones substituted with an ionic moiety, a hydrophilic moiety or both such as 4-trimethylaminomethyl benzophenone hydrochloride or benzophenone sodium 4-methanesulfonate; benzoin C₁-C₄alkyl ether such as benzoin methyl ether; thioxanthones substituted with an ionic moiety, a hydrophilic moiety or both such as 3-(2-hydroxy-3-trimethylaminopropoxy) thioxanthone hydrochloride, 3-(3-trimethylaminopropoxy) thioxanthone hydrochloride, thioxanthone 3-(2-ethoxysulfonic acid) sodium salt or thioxanthone 3-(3-propoxysulfonic acid) sodium salt; or phenyl ketones such as 1-hydroxycyclohexylphenyl ketone, (2-hydroxy-2-propyl)(4-diethylene glycol phenyl)ketone, (2-hydroxy-2-propyl)(phenyl-4-butanecarboxylate)ketone; or commercial products such as Darocure™ or Irgacure™ types, e.g. Darocure 1173 or Irgacure 2959.

The photoinitiator is present in an amount of for example 0.05 to about 1.5% by weight, preferably 0.1 to 1.0% by weight and particularly preferably 0.08 to 0.5% by weight, based on the prepolymer content in each case.

A suitable porogen for use in the present polymerization process may be selected preferably from the range of optionally substituted (i.e. unsubstituted or substituted) poly(alkylene)glycols, preferably those having up to 7 carbon atoms in each alkylene unit which may be the same or different. Unsubstituted poly(alkylene)glycols are preferred. Preferably the porogen is one or more poly(lower alkylene)glycol, wherein lower alkylene in this context denotes alkylene of 2, 3 or 4 carbon atoms, preferably 2 or 3 carbon atoms, in each alkylene unit. The particularly preferred porogens are polyethylenglycols or polypropyleneglycols. The porogens may be of varying molecular weight and are preferably less than 4000 in weight average molecular weight, even more preferred from 300 to 3000 in weight average molecular weight. Substituted poly(alkylene)glycols are understood to include poly(alkylene)glycols wherein one or two hydroxy groups have been replaced by an ether group, e.g. a C₁-C₄-alkoxy group, or an ester group, e.g. a C₁-C₄-alkylcarbonyloxy group, such that a substituted poly(alkylene)glycol may be preferably represented by a mono-poly(alkylene)glycol-ether, a di-poly(alkylene)glycol-ether, a mono-(poly)alkylene)glycol-ester, a di-poly(alkylene)glycol ester, or a poly(alkylene)glycol-monoether-monoester. Preferably, the cell growth substrate polymers of the invention are prepared in the absence of a porogen.

Standard methods well known in the art for effecting polymerization may be utilized, with free radical polymerization being preferred. Free radical polymerization can be simply carried out by radiating (using ultra-violet light) the composition comprising the polymerizable component a photoinitiator and optionally a solvent and/or a porogen in an appropriate container or vessel. The mixture is irradiated for a sufficient time to enable polymerization between monomers to take place. Alternatively, redox initiation or thermal initiation using a thermal initiator such as azobisisobutyronitrile, can be employed.

By way of example, in the manufacture of cell growth materials and implants of such polymers, the appropriate quantities of polymerizable monomers or macromers, solvents and photoinitiator (e.g. Darocure 1173) are mixed together to form a polymerization mixture. The polymerization mixture is then flushed with nitrogen and the required quantity dispensed into an appropriate mold. The mold is closed and clamped and the assembly is placed into an irradiation cabinet equipped with 365 nm UV lamps. The irradiation is performed for the required time and then the halves of the mold are separated. The polymerized material is extracted either in an appropriate solvent, or manually, or by using a special apparatus. The demolded polymerized material is placed inside a perforated cage, the cage then immersed in a beaker containing an appropriate solvent (e.g. water, methanol or a mixture thereof) and gently stirred overnight with occasional replacement of the solvent. If a pore-generating macromer was used in the formulation, the final extraction is carried out with ice water.

As would be obvious to one skilled in the art, the polymerization can also be carried out on the surface of another substrate or within a supporting matrix, so that the substrate is coated with the polymer as herein defined.

With suitable selection, the resultant cell growth substrate is optically transparent, having a refractive index that provides a good match with aqueous media, tissue and cellular material. As a result this polymer is ideal for use as an ocular prostheses, such as corneal onlay or implant. The polymers according to the invention may be formed into other useful cell growth substrates using conventional molding and processing techniques as are well known in the art.

The polymers of the invention are characterized in particular by a high biocompatibility, biostability, non-cytotoxicity, cell growth capability and antifouling properties. Said properties make them suitable as materials for the attachment and growth of human or animal cells in vivo or in vitro, medical implants (such as implantable semipermeable membrane materials, tissue implants in cosmetic surgery, implants containing hormone secreting cells such as pancreatic islet cells, breast implants, artificial joints and the like), in artificial organs, tissue culture apparatus (such as bottles, trays, dishes and the like), in biological reactors (such as those used in the production of valuable proteins and other components by cell culture), in optical instruments, such as microscope slides and the like. As sulfonates possess antifouling properties the polymers of this invention are especially suitable for materials that are designed for long-term implantation.

Ocular prostheses, such as corneal implants, may be made by polymerization in molds and, optionally, the resultant polymer may be fabricated or machined to the desired conformation. Ocular prostheses may be made by other methods which are well known per se to those skilled in the art. Porosity may be provided as described above.

Corneal implants may be placed by way of conventional surgical techniques beneath, within, or through corneal epithelial tissue, or within the corneal stroma or other tissue layers of the cornea. Such implants may change the optical properties of the cornea (such as to correct visual deficiencies) and/or change the appearance of the eye, such as pupil coloration. A corneal implant may comprise an optical axis region which on implantation covers the pupil and provides visual acuity, and a less transparent region which surrounds the periphery of the optical axis region. Alternatively the implant may have the same visual acuity across its dimensions.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

The present invention is further described in the following non-limiting examples. If not other wise specified, all parts are by weight. Temperatures are in degrees Celsius. Molecular weights of macromers or polymers are weight average molecular weights if not otherwise specified.

### EXAMPLE 1

An amount of 2 g styrenesulfonate sodium salt, 1.26 g 2-hydroxyethylmethacrylate, 0.16 g ethyleneglycoldimethacrylate, 0.016 g Darocure 1173 and 4 ml deionized water are mixed and degassed by a stream of argon through the mixture for 30 minutes. 100 μl of the mixture is then dispensed via a syringe into one polypropylene mold. The mold is closed and the mixture cured under UV light for 10 minutes at 6 mW/cm². After opening the mold the cured material is removed and extracted for 9 hours in sterile water and 16 hours in absolute ethanol. Optical clear samples are obtained, which are equilibrated into water and autoclaved. CGI and Cell outgrowth tests reveal no cytotoxicity and an excellent attachment and growth of fibroblast cells.

In analogy to Example 1 the formulations of the following Table 1 are used to generate hydrogels.

TABLE 1
(amounts in grams)

| Ex. | Sulfo 1 | Sulfo 2 | PVP | PEG | HEMA | EGDMA | H₂O | Darocure | NVP |
|---|---|---|---|---|---|---|---|---|---|
| 1.1 | 2.00 | — | — | — | — | 0.17 | 4.00 | 0.02 | 1.09 |
| 1.2 | 1.00 | — | — | — | 2.00 | 0.60 | 1.50 | 0.03 | — |
| 1.3 | 1.00 | — | — | 2.00 | 2.00 | 0.30 | 1.50 | 0.03 | — |
| 1.4 | 0.60 | — | 2.00 | — | 2.00 | 0.30 | 1.60 | 0.03 | — |
| 1.5 | — | 0.16 | — | — | 2.00 | 0.05 | 2.18 | 0.03 | — |
| 1.6 | — | 0.22 | — | — | 2.00 | 0.05 | 2.25 | 0.04 | — |
| 1.7 | — | 0.35 | — | — | 2.00 | 0.06 | 2.30 | 0.04 | — |

Sulfo 1 = Styrenesulfonate sodium salt;
Sulfo 2 = Sodium methallyl sulfonate;
PVP = Poly-N-vinylpyrrolidone;
PEG = Polyethyleneglycol 2000;
HEMA = 2-hydroxyethyl methacrylate;
EGDMA = Ethyleneglycol dimethacrylate;
Darocure = Darocure 1173;
NVP = N-vinylpyrrolidone.

The hydrogels generated from the formulations of Table 1 are in each case transparent and have an excellent cell growth capability.

What is claimed:

1. A cell growth substrate polymer obtainable by polymerizing a polymerizable component comprising from about 30 to about 99% by weight of at least one hydrophilic monomer or macromer which is devoid of a sulfo group, from about 1 to about 70% by weight of at least one sulfo-group-containing monomer, and from 0 to about 20% by weight of at least one low molecular weight crosslinker.

2. A cell growth substrate polymer according to claim 1, wherein the polymerizable component comprises a hydrophilic monomer selected from the group consisting of hydroxyethylmethacrylate (HEMA), N-vinylpyrrolidone (NVP), acrylamide, N,N-dimiethylacrylamide (DMA) and N-acryloylmorpholine.

3. A cell growth substrate polymer according to claim 1, wherein the polymerizable component comprises a hydrophilic monomer which is HEMA.

4. A cell growth substrate polymer according to claim 1, wherein the polymerizable component comprises a hydrophilic macromer which is a vinylfunctionalized polyvinyl alcohol.

5. A cell growth substrate polymer according to claim 1, wherein the sulfo-group-containing monomer is selected from the group consisting of methallylsulfonic acid, styrenesulfonic acid, sulfopropylmethacrylate, sulfopropylacrylate, 2-acrylamido-2-methylpropanesulfonic acid, vinyl sulfonic acid, and a suitable salt thereof.

6. A cell growth substrate polymer according to claim 1, wherein the low molecular weight crosslinker is selected from the group consisting of an ethylenglycol diacrylate or dimethacrylate, di-, tri- or tetraethylenglycol diacrylate and dimethacrylate, allyl (meth)acrylate, a C₂-C₈-alkylene diacrylate and dimethacrylate, divinyl ether, divinyl sulfone, di- and trivinylbenzene, trimethylolpropane triacrylate and trimethacrylate, pentaerythritol tetraacrylate and tetramethacrylate, bisphenol A diacrylate and dimethacrylate, methylene bisacrylamide and -bismethacrylamide, ethylene bisacrylamide and ethylene bismethacrylamide, triallyl phthalate and diallyl phthalate.

7. A cell growth substrate polymers according to claim 1, which is prepared from a polymerizable component comprising 70 to 95% by weight of one or more hydrophilic monomers or macromers, 5 to 20% by weight of a sulfomonomer and 1 to 10% by weight of a crosslinker.

8. A process for the manufacture of a cell growth substrate polymer comprising the steps: (a) forming a composition comprising (i) a polymerizable component comprising from about 20 to about 99% by weight of at least one hydrophilic monomer or macromer which is devoid of a sulfo group, from about 1 to about 70% by weight of at least one sulfo-group-containing monomer, and from 0 to about 79% by weight of at least one low molecular weight crosslinker, and optionally (ii) a solvent and/or a further additive; and (b) polymerizing said composition.

9. A process according to claim 8, wherein in step (b) the composition of step (a) is photopolymerized in the presence of a photoinitiator.

10. A molding obtainable by carrying out the process according to claim 8 in a mold.

11. A molding according to claim 10, which is a biomedical device.

12. A molding according to claim 11, which is a medical implant.

13. A molding according to claim 10, which is an ocular prostheses.

14. A molding according to claim 13, which is an implantable intraocular lens or artificial cornea.
